(19) United States
(12) Patent Application Publication
(10) Pub. No.: US 2025/0356958 A1
(43) Pub. Date: Nov. 20, 2025

(54) METHOD OF PREDICTING MS/MS SPECTRA AND PROPERTIES OF CHEMICAL COMPOUNDS

(71) Applicant: THE TRUSTEES OF INDIANA UNIVERSITY, Bloomington, IN (US)

(72) Inventors: Haixu TANG, Bloomington, IN (US); Yuhui HONG, Bloomington, IN (US); Sujun LI, Zionsville, IN (US)

(21) Appl. No.: 18/872,658

(22) PCT Filed: Jun. 6, 2023
(86) PCT No.: PCT/US2023/024578

§ 371 Date: Dec. 6, 2024

Related U.S. Application Data
(60) Provisional application No. 63/349,329, filed on Jun. 6, 2022.

(51) Int. Cl. G16C 20/30; G16C 20/70

(52) U.S. Cl. CPC G16C 20/30; G16C 20/70

(57) ABSTRACT

Disclosed herein are methods and systems for the prediction of molecular properties from molecular 3-dimensional (3D) conformers. The method comprises receiving the compound information: generating a 3D molecular input point set from the compound information, wherein each atom point of the 3D molecular input point set comprises x, y, z-coordinates and one or more attributes: convoluting the 3D molecular input point set to generate a layer: generating one or more additional layers by repeating the convolution step: encoding the chemical compound by stacking the generated layers; and generating a report comprising one or more predicted properties of the encoded chemical compound.

### CROSS-REFERENCE TO RELATED APPLICATIONS

This application claims benefit of priority to U.S. Patent Application No. 63/349,329, filed Jun. 6, 2022, the contents of which are incorporated by reference in its entirety.

### STATEMENT REGARDING FEDERALLY SPONSORED RESEARCH

This invention was made with government support under 1916645 awarded by the National Science Foundation. The government has certain rights in the invention

### BACKGROUND OF THE INVENTION

Tandem mass (MS/MS) spectrometry is an essential technology for identifying and characterizing chemical compounds at high sensitivity and throughput, and thus is commonly adopted in metabolomics, natural product discovery, and environmental chemistry. However, computational methods for automated compound identification from their MS/MS spectra are still limited, especially for the novel compounds that have not been previously characterized. Accordingly, there is a need for new methods for predicting molecular properties such as mass spectra.

### BRIEF SUMMARY OF THE INVENTION

Disclosed herein are methods and systems for the prediction of molecular properties from molecular 3-dimensional (3D) conformers. The methods described herein utilize an elemental operation on three dimensional (3D) molecular conformers that allow an efficient deep neural network to predict the molecular properties.

One aspect of the invention provides for a method that comprises generating a 3D molecular input point set from compound information, wherein each atom point of the 3D molecular input point set comprises x, y, z-coordinates and one or more attributes; convoluting the 3D molecular input point set to generate a layer, wherein convoluting an input feature matrix generates a dₒᵤₜ×n feature matrix, where the input feature matrix is a dᵢₙ×n feature matrix, n is the number of atoms in the compound, and dᵢₙ comprises the x, y, z-coordinates and the one or more attributes; generating one or more additional layers by repeating the convolution step using the dₒᵤₜ×n feature matrix as the input matrix; encoding the chemical compound by stacking the generated layers; and generating a report comprising one or more predicted properties of the encoded chemical compound. In some embodiments, the encoded chemical compound is permutation invariant.

In some embodiments, each generated layer comprises three subnetworks for atom feature extraction, neighbor feature extraction, and feature integration. In some embodiments, for each atom i with an input feature vector xᵢ (xᵢ∈ [symbol] ), a local subgraph is built for each atom that contains its k-nearest neighbors, whose feature vectors are denoted by yᵢ^{j} (j=1, 2, . . . , k); through the neighbor feature extraction subnetwork, the k neighbor features (bᵢ^{j}, j=1, 2, . . . , k) are derived from the atom features xᵢ and the neighbor features yᵢ^{j}, and then concatenated to obtain a neighbor feature vector cᵢ by using a pooling operation (Σ); through the atom feature extraction subnetwork, the atom feature vector aᵢ is derived from the atom features xᵢ; and through the feature integration subnetwork, the atom and neighbor features are integrated into a latent feature vector xᵢ′ (xᵢ′∈ [symbol] ). In some embodiments, the one or more attributes comprises one or more of encoding of an atom type, number of immediate neighbors, valence, atomic mass, atomic charge, number of immediate hydrogen, aromaticity, and ring system.

In some embodiments, the method comprises multiplying an affine transformation matrix onto the x, y, z-coordinates prior to convolution. Multiplying the affine transformation matrix onto the x, y, z-coordinates may generate a rigid transformation invariant matrix

In some embodiments, the encoded chemical compound is combined with meta data. Exemplary meta data may comprise a precursor type or a collision energy.

In some embodiments, the report is generated by embedding the encoded chemical compound into a vector by fully connected and/or max-pooling layers. In some embodiments, the report comprises a predicted mass spectra mass-to-charge-ratio (m/z) or a relative intensity at the predicted m/z.

In some embodiments, pretrained prediction model weights are used to initialize weights for a second, different prediction model. Exemplary pretrained prediction model weights may be mass spectrometry prediction model weights. The report may comprise a predicted chemical property that is neither a mass spectra mass-to-charge-ratio (m/z) nor a relative intensity at the predicted m/z.

Systems and computer readable media for implementing the methods described herein are also provided for.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 illustrates a method for predicting one or more properties of a chemical compound.

FIG. 2 illustrates the distribution of atom types and precursor types.

FIG. 3 illustrates the convolution operation of MolConv.

FIG. 4 illustrates the architecture of Mol3DNet.

FIG. 5 illustrates compounds from MS/MS libraries.

FIG. 6 illustrates spectrum prediction results comparing with CFM-ID 4.0.

FIG. 7 illustrates an exemplary prediction system.

### DETAILED DESCRIPTION OF THE INVENTION

Disclosed herein are methods and systems for the prediction of molecular properties from molecular 3-dimensional (3D) conformers. The methods described herein utilize an elemental operation, named “MolConv,” on three dimensional (3D) molecular conformers, from which a an efficient deep neural network, named “Mol3DNet,” was developed to predict the molecular properties, including tandem mass spectrometry (MS/MS) spectra of chemical compounds. The model may be trained using MS/MS spectra in public spectral libraries, including NIST20, GNPS, and MoNA. The Examples demonstrate that the transfer learning between the MS/MS spectra acquired by using different mass spectrometry instruments and fragmentation methods improves the prediction accuracy significantly. When evaluated on the testing dataset consisting of experimental spectra that were not used for the training purpose, the disclosed methods achieves state-of-the-art performance. The Examples demonstrate cosine similarities between the predicted and experimental spectra are 0.549 and 0.621, respectively, for the Higher-energy collisional dissociation (HCD) spectra (acquired using the ion trap MS instruments) and the combination of Q-TOF spectra (acquired using the quadrupole/time-of-flight MS instruments) and QqQ spectra (acquired using the triple-quadrupole MS instruments).

Moreover, the Examples further demonstrate that the representation learned in spectra prediction can be transferred to improving the prediction of diverse chemical properties of compounds which are also used for compound identification. For instance, the Examples demonstrate the transfer learning from spectra prediction to exemplary chemical properties, such as retention time, collision cross section (CCS), solubility, and toxicity.

Because of its high sensitivity and throughput, mass spectrometry (MS) coupled with gas chromatography (GC) or liquid chromatography (LC) has long been adopted for the characterization and structural elucidation of chemical compounds. Liquid chromatography tandem mass spectrometry (LC-MS/MS), which detects the fragment ions of compounds resulting from the high energy collision in a collision cell, becomes an essential technology for identifying and quantifying chemical compounds in complex samples in multiple application areas including metabolomics, natural product discovery, and environmental chemistry. For instance, metabolomics aims to identify and quantify metabolites present in tissues and body fluids, leading to the discovery of molecular biomarkers associated with diseases and clinical conditions. In untargeted metabolomics, LC-MS/MS is used to acquire thousands of MS/MS spectra in a single sample, from which metabolites are to be identified. Many MS-based metabolite identification systems exploited the spectra searching against a reference spectral library (RSL) consisting of the MS/MS spectra of previously identified chemical compounds. In practice, however, compound spectra in the available spectral libraries (e.g., NIST20, HMDB, MassBank, and GNPS) are limited, and thus a majority (up to 80%) of MS/MS spectra in metabolomic experiments remain unidentified by the spectral library searching methods. Compound identification remains a big obstacle in the other applications of LC-MS/MS such as environmental chemistry and natural product discovery, in which the fraction of unknown compounds in a target sample is even greater.

The disclosed technology utilizes an efficient deep neural network, Mol3DNet, based on an elemental operation of MolConv on the three dimensional (3D) molecular conformers of compounds to predict the MS/MS spectra of chemical compounds. In Mol3DNet, a 3D conformer is represented as a point set. The molecular point set encodes accurate 3D coordinates and attributes of the atoms, and the chemical bonds are represented as neighboring vectors. When trained and tested on the MS/MS spectra of chemical compounds from several spectral libraries, the method achieved higher accuracy and faster speed than CFM-ID 4.0 [Fei Wang, et al. Cfm-id 4.0: more accurate ESI-MS/MS spectral prediction and compound identification. Analytical Chemistry, 93(34):11692-11700, 2021], a hybrid algorithm combining rule-based and machine learning methods.

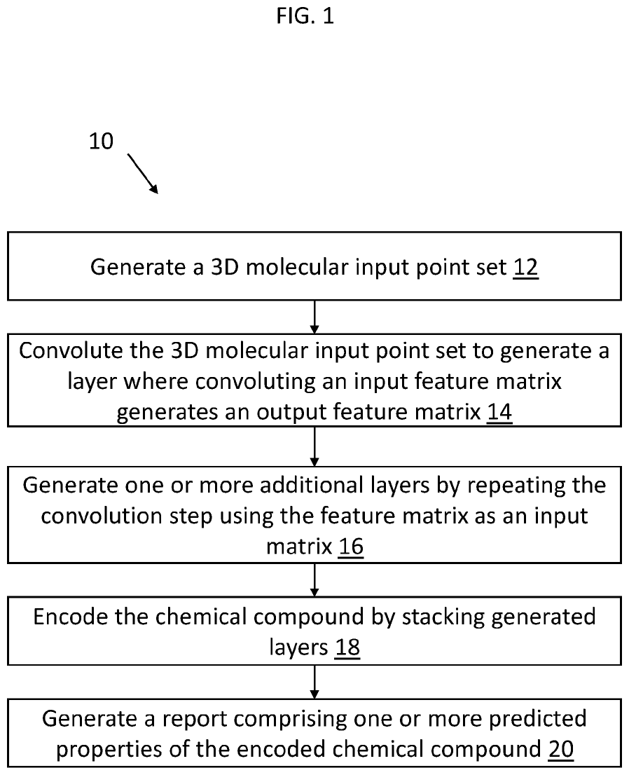

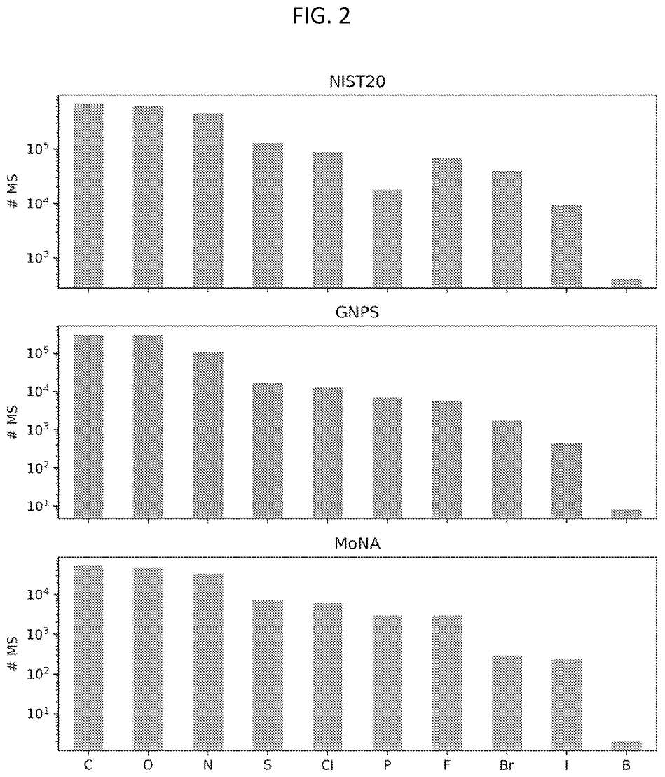

One aspect of the technology comprises a method for generating a report comprising one or more predicted properties of an encoded chemical compound. FIG. 1 illustrates the method for predicting one or more properties of a chemical compound 10.

Although the Examples demonstrate the use of mass spectra data as a training data set in the described methods other chemical training data sets such as NMR spectroscopy, circular dichroism (CD), or Raman spectroscopy may also be used. Additionally, the Examples demonstrate the use of mass spectra data as a training data set for the transfer of representation learning for the prediction of a second, different prediction model, e.g., different mass spectrometry methods, retention time, collisional cross section, solubility, reactivity, and toxicity, other chemical properties may also be predicted.

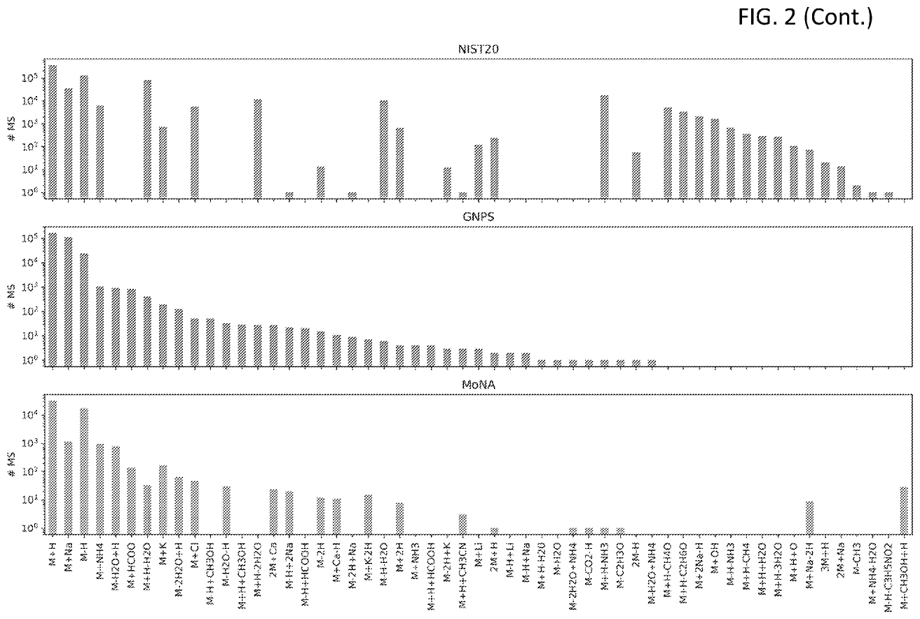

By way of example, MS/MS spectra of chemical compounds was collected from NIST20[Xiaoyu Yang, et al Extending a tandem mass spectral library to include ms2 spectra of fragment ions produced in-source and msn spectra. Journal of The American Society for Mass Spectrometry, 28(11):2280-2287, 2017.], GNPS [Mingxun Wang, et al. Sharing and community curation of mass spectrometry data with global natural products social molecular net-working. Nature biotechnology, 34(8):828-837, 2016.], and MassBank of North America (MoNA) [Hisayuki Horai, et al. Massbank: a public repository for sharing mass spectral data for life sciences. Journal of mass spectrometry, 45(7):703-714, 2010.], including those acquired by using high-energy collisional dissociation (HCD), quadrupole time-of-flight (Q-TOF) or triple-quadrupole (QqQ) MS instruments. They are pre-possessed by following steps: (1) The missing isomeric SMILES are fixed by searching with the synonyms names in PubChem [Sunghwan Kim, et al. Pubchem in 2021: new data content and improved web interfaces. Nucleic acids research, 49 (D1): D1388-D1395, 2021.]. (2) The mass spectra has less than 5 peaks are filtered out, because they are unreliable. (3) The m/z range is limited in 0 1500, because few spectra have m/z above 1500. (4) The molecules composite by high-frequency atoms (C, H, O, N, F, S, CI, P, B, Br, I) are maintained. (5) The spectra with high-frequency precursor types ([M +H]+, [M H]−, [M+Na]+, etc.) are retained. The summary statistics for the libraries used in our experiments are shown in Table 1. The distribution of atoms and precursor types are summarized in FIG. 2. For training and testing purposes, we combined the Q-TOF and QqQ spectra together because these two types of spectra from the same compounds are very similar.

TABLE 1
Statistics of Tandem Mass Spectra Libraries

| Dataset | Instrument Type | # Mass Spectra | # Compounds |
|---|---|---|---|
| GNPS | HCD | 0 | 0 |
| | QTOF | 21112 | 4730 |
| | QqQ | 7563 | 1207 |
| | Unknow | 0 | 0 |
| NIST20 | HCD | 535283 | 21037 |
| | QTOF | 30870 | 2167 |
| | QqQ | 21285 | 1700 |
| | Unknow | 0 | 0 |
| MoNA | HCD | 18595 | 1913 |
| | QTOF | 15650 | 2776 |
| | QqQ | 4112 | 707 |
| | Unknow | 7720 | 3861 |

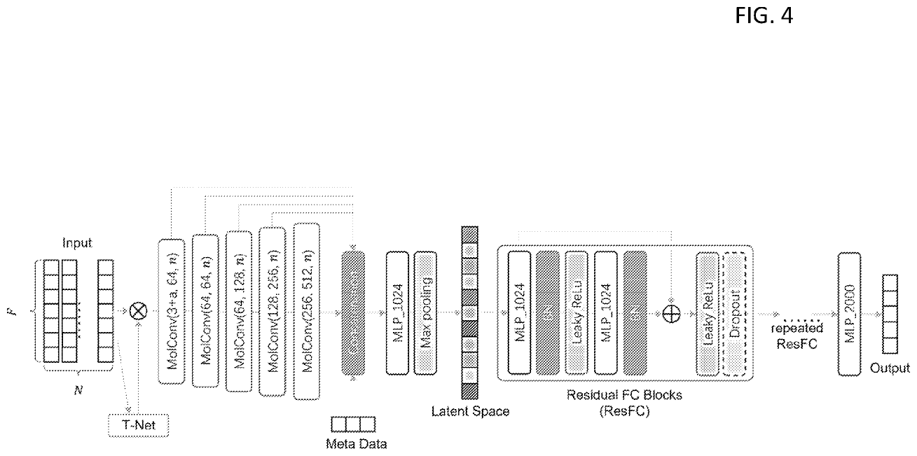

Referring to FIG. 1, a 3D molecular input set is generated 12. In the Examples, Chem.MolFromSmiles( ) and AllChem.EmbedMolecule( ) functions in the RDkit library [Greg Landrum, et al. rdkit/rdkit: 2020 03 1 (q1 2020) release. March. https://doi. org, 10, 2020.] were used to generate the 3D conformer of a compound as a Chem.rdchem.Mol object, which contains the x, y, z-coordinates of each atom as well as the information of chemical bonds, from its SMILES string. As mentioned above, a compound is then encoded into a fixed number of n atom points (i.e., the point set); when the number of atoms is smaller than n, the point set is padded to n points with the coordinates of the padded points set as zeros. Each atom point contains the x, y, z-coordinates and atomic attributes, as shown in Table 2. Atom attributes may be generated by using RDKit. An experimental MS/MS spectra may be represented by a 1D spectral vector, in which each dimension represents the total intensity of fragment ions in a bin of the fixed mass-to-charge-ratio (m/z). Here, the number of bins is dependent on the mass resolution of the MS/MS spectra, and is a flexible hyper-parameter in the model; by default, resolution of 0.2 was used, and thus the spectral vector has 7500 dimensions (within the m z range between 0 and 1500 that covers almost all fragment ions observed in the MS/MS spectra). Finally, the MS experimental conditions were considered, including the collision energy and the precursor types as metadata concatenate to the embedded point set (FIG. 4). The collision energy may be normalized to the range of 0 to 1, and the precursor types can be encoded in one-hot codes. If the collision energy is unlabeled, 0 will be filled.

TABLE 2
Molecular Encoding Information

| Index | Description |
|---|---|
| 0-2 | x, y, z coordinates |
| 3-14 | one-hot encoding of the atom type |
| 15 | number of immediate neighbors who are |
| | “heavy” (nonhydrogen) atoms |
| 16 | valence minus the number of hydrogens |
| 17 | atomic mass |
| 18 | atomic charge |
| 19 | number of implicit hydrogens |
| 20 | is aromatic |
| 21 | is in a ring |

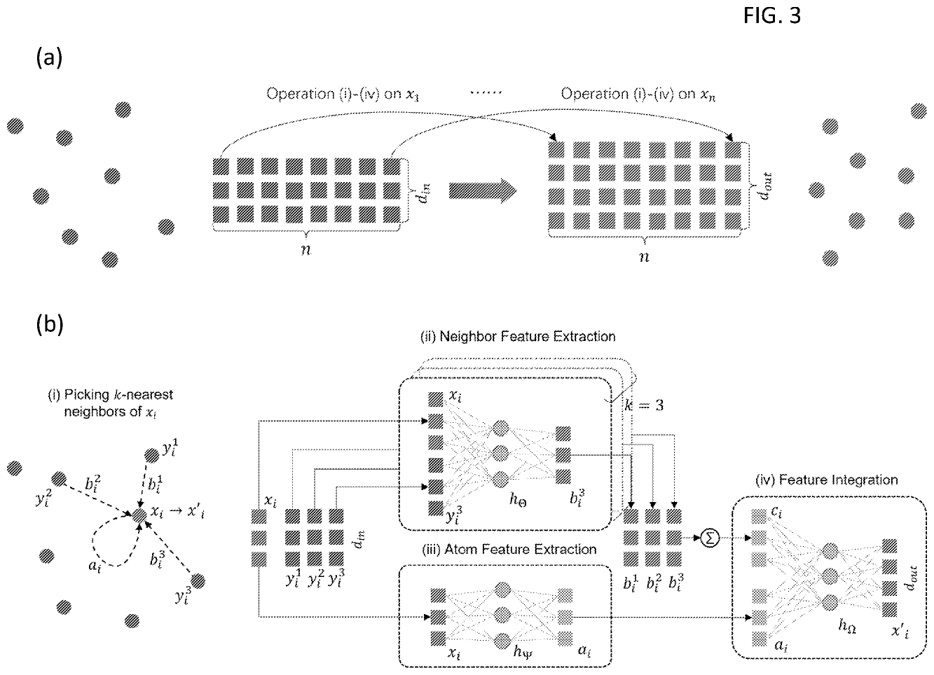

Two principles of operation are necessary for the convolution operations on molecular point sets: permutation invariance and rigid transformation invariance (i.e., the Euclidean transformation invariance). They guarantee that the order of atoms and the rigid transformation of the input molecule will not affect the output of the operation. MolConv (shown in FIG. 3) is designed to satisfy these two conditions. MolConv integrates the features from both the atoms (represented as 3D points) and atomic interactions (e.g., the chemical bonds) in a small molecule.

Again referring to FIG. 1, the 3D molecular input point set is convoluted to generate a layer where convoluting an input feature matrix generates an output feature matrix 14. One or more additional layers may be generated by repeating the convolution step using the output feature matrix an input matrix 16. The chemical compound may be encoded by stacking generated layers 18.

FIG. 3 illustrates the operation of MolConv. Panel (a) shows that multiple layers of MolConv can be stacked sequentially to form an encoder of a chemical compound. Each MolConv layer aims to convert a dᵢₙ×n feature matrix into dₒᵤₜ×n feature matrix, where n is the number of atoms in the compound. In the first MolConv layer of the encoder, an input molecule is represented as a matrix, including n columns of x, y, z-coordinates and other properties of atoms (Table 2). For the subsequent layers, the output matrix of previous layer (i.e., each column representing the latent vector for each of the n atoms) becomes the input of the current layer. Panel (b) shows each MolConv layer consists of three subnetworks for the feature extraction and integration in four steps: 1) for each atom i with the input feature vector xᵢ (xᵢ∈ [symbol] ), a local subgraph is built for each atom that contains its k-nearest neighbors, whose feature vectors are denoted by yᵢ^{j} (j=1, 2, . . . , k); (ii) through the neighbor feature extraction subnetwork, the k neighbor features (bᵢ^{j}, j=1, 2, . . . , k) are derived from the atom features xᵢ and the neighbor features yᵢ^{j}, and then concatenated to obtain the neighbor i feature vector cᵢ by using the pooling operation (Σ); (iii) through the atom feature extraction subnetwork, the atom feature vector aᵢ is derived from the atom features xᵢ; and (iv) finally, through the feature integration subnetwork, the atom and neighbor features are integrated into a latent feature vector xᵢ′ (xᵢ′∈ [symbol] ), as the output of the MolConv layer.

Consider a molecule with n atoms, denoted by X={x1, x2, . . . , xn}⊆ [symbol] . For the first layer, dᵢₙ=21 (shown in Table 2). In a deep neural network architecture, each layer operates on the output of the previous layer, and thus, dᵢₙ varies for different layers. In other words, dᵢₙ is the output feature dimensionality of the previous layer. The general idea of permutation invariance feature extraction is applying a symmetric function on transformed elements in the set:

$$f ( \{ {x}_{1} , {x}_{2} , … , {x}_{n} \} ) \approx g ( h ( {x}_{1} ) , h ( {x}_{2} ) , … , h ( {x}_{n} ) ) \qquad ( 1 )$$

$$where f : \to , h : \to and g : \underbrace{{ℝ}^{K} \times … \times {ℝ}^{K}} \to ℝ .$$

We concretize g as max-pooling and h as:

$${x}_{i}^{′} = {h}_{Ω} ( {a}_{i} , {b}_{i} ) \qquad ( 2 )$$

$${a}_{i} = {h}_{Ψ} ( {x}_{i} ) \qquad ( 3 )$$

$${b}_{i} = {\sum }_{j = 1}^{k} {h}_{θ} ( {x}_{i} , {y}_{i}^{j} ) \qquad ( 4 )$$

where i=1, 2, . . . , n, h_{Ω} is symmetric because h_{ψ}, h_{Θ} and summarizing is symmetric to elements. Hence, our feature extraction method is permutation invariance.

Against referring to FIG. 1, one or more properties of a chemical compound may be predicted that may be provided as a report 20. Based on the elemental operation MolConv, Mol3Dnet, a 3D convolutional neural network, can be constructed as illustrated in FIG. 4. To satisfy the condition of rigid transformation invariance, a mini-neural network called T Net is adopted to learn an affine transformation matrix that is multiplied onto the inputs x, y, z-coordinates. The features from input matrix (point sets) are extracted by MolConv at different scales, which are subsequently concatenated and embedded into a vector by fully connected (FC) and max-pooling layers. In the end, we use the residual fully connected blocks to obtain the final prediction.

Mol3Dnet is a 3D convolutional neural network that uses the MolConv as the elemental convolution operation. The input of the network is the x, y, z-coordinates and attributions of the atoms shaped a n x di matrix, where n denotes the number of atoms in the compound, and the additional input of meta-data includes the precursor types and the collision energy of the mass spectra. The output of the network can be a vector representation of the mass spectrum, and chemical properties of the compound, e.g., the retention time, the collision cross section (CCS), etc.

Focusing on the relative intensities of the fragment ions in the spectra, we used the cosine similarity as the loss function.

$$ℒ = 1 - cos ( y , \hat{y} ) = 1 - \frac{y \cdot \hat{y}}{‖ y ‖ ‖ \hat{y} ‖} \qquad ( 5 )$$

where y represents the experimental mass spectra and ŷ represents the predicted mass spectra.

In Mol3DNet, each compound is embedded into a latent vector by the encoder, indicating the model learned the representation of the input compound that is sufficient to predict the mass spectra of any compound. This molecular representation captures essential structural information about the compounds, which can be transferred to the relevant prediction tasks, such as the prediction of chemical properties of compound. Here, as a proof of concept, the Examples demonstrate this transfer learning approach indeed improve the prediction of the retention time and the collisional cross section (CCS) of compounds. Specifically, the weights in the pretrained spectra prediction models encoder are saved, and the encoder is loaded and initialized as the start point to the new task. When training, the representation learning is tuned by training dataset, and the decoder is trained independently.

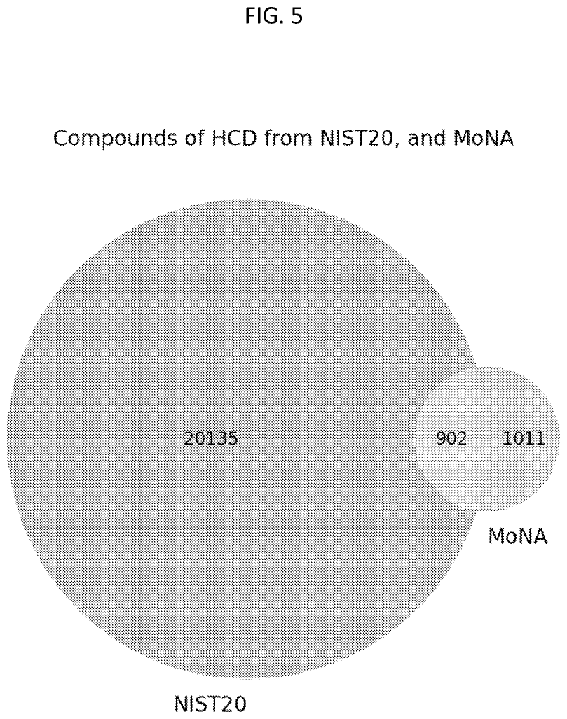

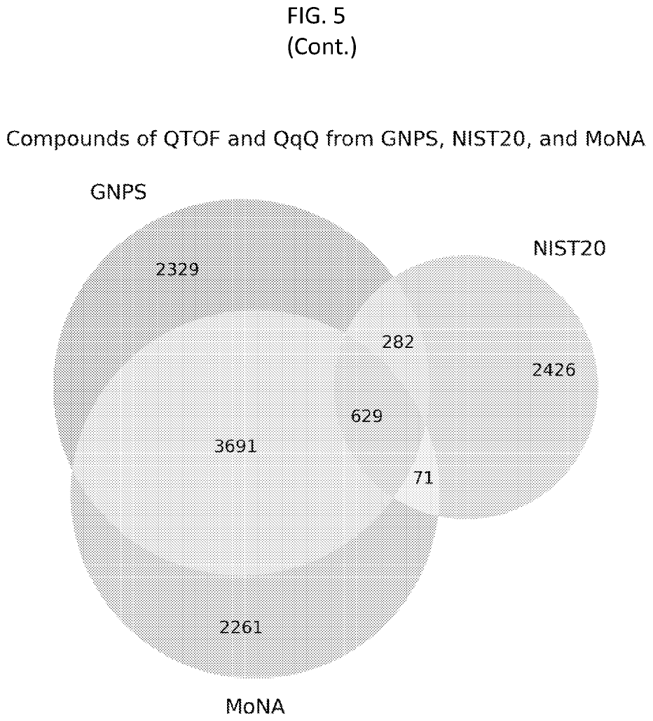

To enlarge the compounds diversity, the mass spectra from the same instrument are merged together. The overlap of libraries are shown in FIG. 5. The overlap compounds have MS/MS in high consistency, whose similarity is higher than 0.8. In the Examples, the unified mass spectra libraries are randomly split into subsets in a ratio of 9:1 for training and test respectively. Cosine similarity measures the prediction accuracy. The datasets size and prediction results are shown in Table 3. The column “Ours” are the results of training independently on each instrument, and the column “Ours-TL” shows the results of training with transfer learning from HCD to QTOF. The results indicate that the molecular representation learning from HCD libraries can be transferred into the QTOF mass spectra prediction. With this transfer learning, the accuracy of QTOF mass spectra prediction is improved significantly.

TABLE 3
Spectrum Prediction Results on All Precursor Types

| Dataset | Instrument | # MS | # MOL | Ours | Ours-TL |
|---|---|---|---|---|---|
| NIST20 | HCD | 535,283 | 21,037 | 0.539 | — |
| MoNA | HOD | 18,595 | 1,913 | 0.551 | — |
| GNPS | QTOF, QqQ | 39,525 | 6,931 | 0.538 | 0.607 |
| NIST20 | QTOF, QqQ | 50,944 | 3,408 | 0.558 | 0.648 |
| MoNA | QTOF, QqQ, Unknow | 28,161 | 6,652 | 0.567 | 0.617 |

To compare with the previous methods, our model was evaluated on positive [M+H]+ ionization and negative [M−H]-ionization modes (shown in FIG. 6 and Table 4). All the HCD results are from the training independent model, and all the QTOF results are from the transfer learning model. In CFM-ID, they predict the mass spectra in three-level collision energies (10 eV, 20 eV, and 40 eV). The best prediction in those levels was chosen as the final result. It shows that the disclosed model performs better than CFM-ID in most of the subsets, especially the large subset.

TABLE 4
Spectrum Prediction Results Comparing with CFM-ID 4.0

| | | Test # MS | | CFM-ID 4.0 | | Ours | |
|---|---|---|---|---|---|---|---|
| | | [M⁺ | [M − | [M⁺ | [M − | [M⁺ | [M − |
| Dataset | Instrument | H]⁺ | H]⁻ | H]⁺ | H]⁻ | H]⁺ | H]⁻ |
| NIST20 | HCD | 27,493 | 26,369 | 0.541 | 0.416 | 0.564 | 0.514 |
| MoNA | HCD | 1,270 | 548 | 0.615 | 0.537 | 0.611 | 0.411 |
| GNPS | QTOF, QqQ | 2,089 | 1,073 | 0.502 | 0.495 | 0.615 | 0.593 |
| NIST20 | QTOF, QqQ | 1,372 | 217 | 0.567 | 0.583 | 0.666 | 0.580 |
| MoNA | QTOF, QqQ, | 773 | 716 | 0.528 | 0.559 | 0.632 | 0.600 |
| | Unknow | | | | | | |

The disclosed model can also be transferred to chemical properties prediction. In this section, the model on HCD mass spectra prediction was used as a pre-trained model doing transfer learning. To evaluate our model, coefficient of determination (R2), mean absolute error (AE), media absolute error (AE), mean relative error (RE) and media relative error (RE) are used as the metrics. Table 6 shows the performance on Collision Cross Section (CCS) and Retention Time (RT). The model with transfer learning can always get higher R² and lower errors.

TABLE 5
Statistics of Chemical Properties Dataset

| Task | # MOL | Range | Mean ± S.D. |
|---|---|---|---|
| CCS | 2,193 | [105.900, 322.500] | 109.512 ± 36.799 |
| RT | 80,038 | [0.300, 1471.700] | 790.111 ± 206.651 |

TABLE 6
Chemical Properties Regression Results

| Task | Model | R² | Mean AE | Media AE | Mean RE | Media RE |
|---|---|---|---|---|---|---|
| CCS | Ours | 0.957 | 6.014 | 4.629 | 0.035 | 0.028 |
| | Ours-TL | 0.961 | 5.030 | 3.633 | 0.029 | 0.020 |
| RT | Ours | 0.778 | 58.459 | 32.061 | 0.095 | 0.042 |
| | Ours-TL | 0.787 | 55.300 | 31.651 | 0.092 | 0.041 |

To further demonstrate the use of transfer learning, Table 7 shows the result of solubility prediction. Similar as the method for predicting the elution time and CCS of peptides, here, the spectra prediction model was tuned using the water solubility of peptides assembled in the database of AqSolDB [Sorkun, Murat Cihan, Abhishek Khetan, and Süleyman Er. “AqSolDB, a curated reference set of aqueous solubility and 2D descriptors for a diverse set of compounds.” Scientific data 6, no. 1 (2019): 1-8]. The whole dataset was randomly partitioned into the training (80%) and testing (20%) data, and the model was first re-trained by using the training data and then evaluated on the testing data to ensure there is no information leak in the testing process.

TABLE 7
Solubility Properties Regression Results

| | # MOL | Range | Mean ± S.D. | R\hat{ }2 |
|---|---|---|---|---|
| SOL | 9,041 | [−13.171, 2.137] | −2.951 ± 2.324 | 0.811 |
| SOL TL | 9,041 | [−13.171, 2.137] | −2.951 ± 2.324 | 0.824 |
| | Mean | | | Median |
| | Absolute | Median Absolute | Mean Relative | Relative |
| | Error | Error | Error | Error |
| SOL | 0.710 | 0.506 | 0.395 | 0.206 |
| SOL TL | 0.678 | 0.487 | 0.383 | 0.184 |

SOL: Solubility
TL: Transfer Learning

To further demonstrate the use of transfer learning, Table 8 shows the result of toxicity prediction. Again, here the transfer learning was achieved by fine-tuning the spectra prediction model using the toxicity data collected by the TorchDrug project [https://torchdrug.ai/docs/api/datasets.html#molecule-property-prediction-datasets]. And the training and evaluation was performed on the 4:1 partition of each dataset as described above.

TABLE 8
Solubility Properties Regression Results

| | | | active | DeepTox | | Our |
|---|---|---|---|---|---|---|
| Assay | Active | Inactive | % | [5] | Ours | TL |
| NR-AR | 261 | 7155 | 3.52% | 0.346 | 0.844 | 0.905 |
| NR-AR-LDB | 220 | 6686 | 3.19% | 0.929 | 0.801 | 0.875 |
| NR-AhR | 742 | 5927 | 11.13% | 0.841 | 0.822 | 0.791 |
| NR Aromatase | 285 | 5652 | 4.80% | 0.792 | 0.786 | 0.802 |
| NR-ER | 662 | 5533 | 10.69% | 0.695 | 0.728 | 0.721 |
| NR-ER-LBD | 303 | 6778 | 4.28% | 0.727 | 0.755 | 0.770 |
| NR-PPAR- | 175 | 6415 | 2.66% | 0.710 | 0.758 | 0.815 |
| gamma | | | | | | |
| SR-ARE | 919 | 4988 | 15.56% | 0.802 | 0.719 | 0.747 |
| SR-ATADS | 243 | 6986 | 3.36% | 0.796 | 0.750 | 0.760 |
| SR-HSE | 337 | 6236 | 5.13% | 0.810 | 0.663 | 0.721 |
| SR-MMP | 906 | 4997 | 15.35% | 0.849 | 0.807 | 0.871 |
| SR-p53 | 411 | 6497 | 5.95% | 0.749 | 0.761 | 0.748 |
| Assay_AVG | — | — | — | 0.754 | 0.766 | 0.794 |

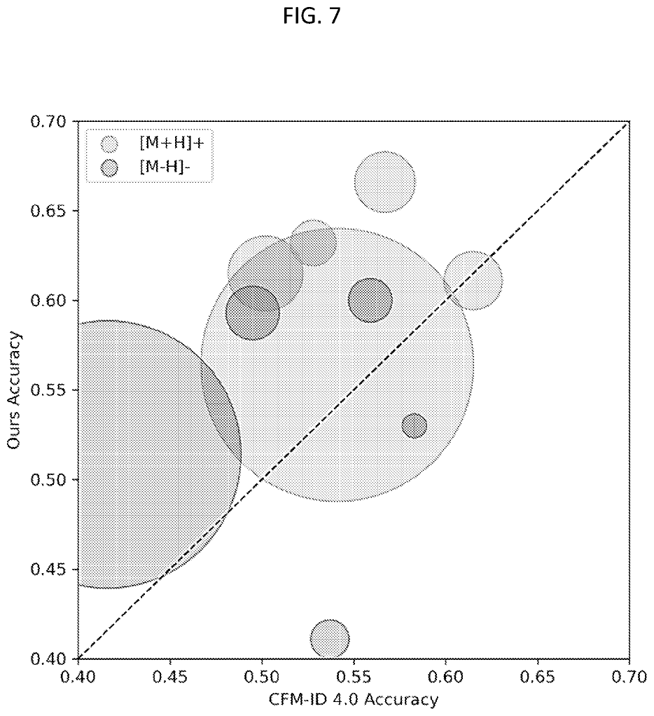

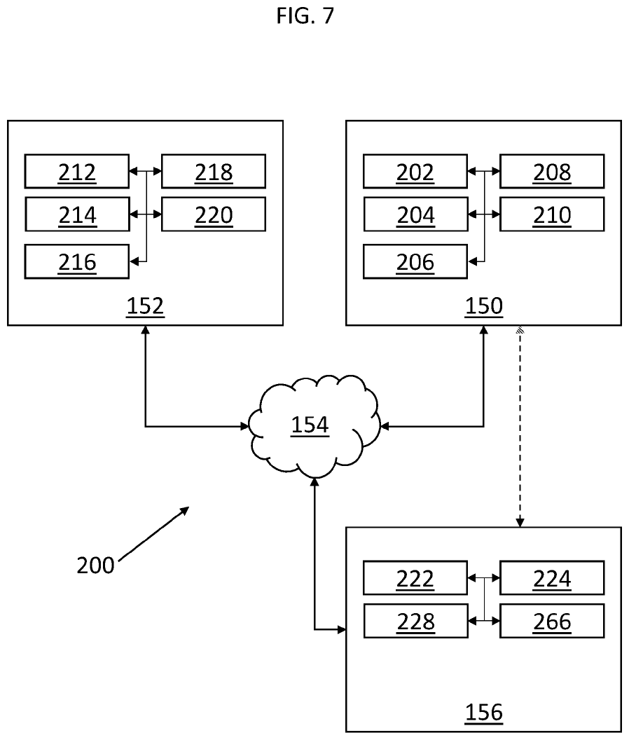

Referring now to FIG. 7, an example of a system 200 for predicting MS/MS spectra and other properties of chemical compounds in accordance with some embodiments of the systems and methods described in the present disclosure is shown. As shown in FIG. 7, a computing device 150 can receive one or more types of data (e.g., compound information related to a chemical compound) from a data source 156 and/or input 202. In some embodiments, computing device 150 can execute at least a portion of a method for predicting one or more properties of a chemical compound 100 as exemplified in FIG. 7.

Additionally or alternatively, in some embodiments, the computing device 150 can communicate information about data received from the data source 156 or input 202 to a server 152 over a communication network 154, which can execute at least a portion of method 100. In such embodiments, the server 152 can return information to the computing device 150 (and/or any other suitable computing device) indicative of a report comprising one or more predicted properties of the encoded chemical compound.

In some embodiments, computing device 150 and/or server 152 can be any suitable computing device or combination of devices, such as a desktop computer, a laptop computer, a smartphone, a tablet computer, a wearable computer, a server computer, a virtual machine being executed by a physical computing device, and so on.

In some embodiments, data source 152 can be any suitable source of data (e.g., chemical information, pretrained prediction model weights, 3D confirmation data, atom type, number of immediate neighbors, position of immediate neighbors, valence, atomic mass, atomic charge, number of immediate hydrogen, aromaticity, ring system, spectral information, and so forth), another computing device (e.g., a server storing data), and so on. In some embodiments, data source 156 can be local to computing device 150. For example, data source 156 can be incorporated with computing device 150 (e.g., computing device 150 can be configured as part of a device for measuring, recording, estimating, acquiring, or otherwise collecting or storing data). As another example, data source 156 can be connected to computing device 150 by a cable, a direct wireless link, and so on. Additionally or alternatively, in some embodiments, data source 156 can be located locally and/or remotely from computing device 150, and can communicate data to computing device 150 (and/or server 152) via a communication network (e.g., communication network 154).

In some embodiments, a user provides the computing device 150 some or all of the compound information used in the methods described herein. Where a user provides incomplete compound information, the computing device 150 may retrieve additional compound information from locally stored compound information, the server 152, data source 156, or any combination thereof.

In some embodiments there the server 152 performs all of or a portion of the methods described herein, the server 152 may retrieve additional compound information from locally stored compound information, the computing device 150, data source 156, or any combination thereof. In some embodiments, communication network 154 can be any suitable communication network or combination of communication networks. For example, communication network 154 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 154 can be a local area network, a wide area network, a public network (e.g., the Internet), a private or semi-private network (e.g., a corporate or university intranet), any other suitable type of network, or any suitable combination of networks. Communications links shown in FIG. 7 can each be any suitable communications link or combination of communications links, such as wired links, fiber optic links, Wi-Fi links, Bluetooth links, cellular links, and so on.

An example of hardware 200 that can be used to implement data source 156, computing device 150, and server 152 in accordance with some embodiments of the systems and methods described in the present disclosure is shown. As shown in FIG. 7, in some embodiments, computing device 150 can include a processor 202, a display 205, one or more inputs 206, one or more communication systems 208, and/or memory 210. In some embodiments, processor 202 can be any suitable hardware processor or combination of processors, such as a central processing unit (“CPU”), a graphics processing unit (“GPU”), and so on. In some embodiments, display 1204 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 1206 can include any suitable input devices and/or sensors that can be used to receive user input, such as a keyboard, a mouse, a touchscreen, a microphone, and so on.

In some embodiments, communications systems 208 can include any suitable hardware, firmware, and/or software for communicating information over communication network 154 and/or any other suitable communication networks. For example, communications systems 208 can include one or more transceivers, one or more communication chips and/or chip sets, and so on. In a more particular example, communications systems 208 can include hardware, firmware, and/or software that can be used to establish a Wi-Fi connection, a Bluetooth connection, a cellular connection, an Ethernet connection, and so on.

In some embodiments, memory 210 can include any suitable storage device or devices that can be used to store instructions, values, data, or the like, that can be used, for example, by processor 202 to present content using display 204, to communicate with server 152 via communications system(s) 208, and so on. Memory 210 can include any suitable volatile memory, non-volatile memory, storage, or any suitable combination thereof. For example, memory 210 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 210 can have encoded thereon, or otherwise stored therein, a computer program for controlling operation of computing device 150. In such embodiments, processor 202 can execute at least a portion of the computer program to present content (e.g., images, user interfaces, graphics, tables), receive content from server 152, transmit information to server 152, and so on. For example, the processor 202 and the memory 210 can be configured to perform the methods described herein (e.g., the method of FIG. 1).

In some embodiments, server 152 can include a processor 212, a display 214, one or more inputs 216, one or more communications systems 218, and/or memory 220. In some embodiments, processor 212 can be any suitable hardware processor or combination of processors, such as a CPU, a GPU, and so on. In some embodiments, display 214 can include any suitable display devices, such as an LCD screen, LED display, OLED display, electrophoretic display, a computer monitor, a touchscreen, a television, and so on. In some embodiments, inputs 216 can include any suitable input devices and/or sensors that can be used to receive user input, such as a keyboard, a mouse, a touchscreen, a microphone, and so on.

In some embodiments, communications systems 218 can include any suitable hardware, firmware, and/or software for communicating information over communication network 154 and/or any other suitable communication networks. For example, communications systems 218 can include one or more transceivers, one or more communication chips and/or chip sets, and so on. In a more particular example, communications systems 218 can include hardware, firmware, and/or software that can be used to establish a Wi-Fi connection, a Bluetooth connection, a cellular connection, an Ethernet connection, and so on.

In some embodiments, memory 220 can include any suitable storage device or devices that can be used to store instructions, values, data, or the like, that can be used, for example, by processor 212 to present content using display 214, to communicate with one or more computing devices 150, and so on. Memory 220 can include any suitable volatile memory, non-volatile memory, storage, or any suitable combination thereof. For example, memory 220 can include RAM, ROM, EPROM, FEPROM, other types of volatile memory, other types of non-volatile memory, one or more types of semi-volatile memory, one or more flash drives, one or more hard disks, one or more solid state drives, one or more optical drives, and so on. In some embodiments, memory 220 can have encoded thereon a server program for controlling operation of server 152.

In such embodiments, processor 212 can execute at least a portion of the server program to transmit information and/or content (e.g., data, images, a user interface) to one or more computing devices 150, receive information and/or content from one or more computing devices 150, receive instructions from one or more devices (e.g., a personal computer, a laptop computer, a tablet computer, a smartphone), and so on.

In some embodiments, the server 152 is configured to perform the methods described in the present disclosure. For example, the processor 212 and memory 220 can be configured to perform the methods described herein (e.g., the method of FIG. 1).

In some embodiments, data source 156 can include a processor 222, one or more data acquisition systems 224, one or more communications systems 226, and/or memory 228. In some embodiments, processor 222 can be any suitable hardware processor or combination of processors, such as a CPU, a GPU, and so on. In some embodiments, the one or more data acquisition systems 224 are generally configured to acquire data. Additionally or alternatively, in some embodiments, the one or more data acquisition systems 224 can include any suitable hardware, firmware, and/or software for coupling to and/or controlling operations of a data acquisition system (e.g., a mass spectrometry system or other system for acquiring data types). In some embodiments, one or more portions of the data acquisition system(s) 224 can be removable and/or replaceable.

Note that, although not shown, data source 156 can include any suitable inputs and/or outputs. For example, data source 156 can include input devices and/or sensors that can be used to receive user input, such as a keyboard, a mouse, a touchscreen, a microphone, a trackpad, a trackball, and so on. As another example. data source 156 can include any suitable display devices, such as an LCD screen, an LED display, an OLED display, an electrophoretic display, a computer monitor, a touchscreen, a television, etc., one or more speakers, and so on.

In some embodiments, communications systems 226 can include any suitable hardware, firmware, and/or software for communicating information to computing device 150 (and, in some embodiments, over communication network 154 and/or any other suitable communication networks). For example, communications systems 226 can include one or more transceivers, one or more communication chips and/or chip sets, and so on. In a more particular example, communications systems 226 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 228 can include any suitable storage device or devices that can be used to store instructions, values, data, or the like, that can be used, for example, by processor 222 to control the one or more data acquisition systems 224, and/or receive data from the one or more data acquisition systems 224; to generate images from data; present content (e.g., data, images, a user interface) using a display; communicate with one or more computing devices 150; and so on. Memory 228 can include any suitable volatile memory, non-volatile memory, storage, or any suitable combination thereof. For example, memory 228 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 228 can have encoded thereon, or otherwise stored therein, a program for controlling operation of data source 156. In such embodiments, processor 222 can execute at least a portion of the program to generate images, transmit information and/or content (e.g., data, images, a user interface) to one or more computing devices 150, receive information and/or content from one or more computing devices 150, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

Unless otherwise specified or indicated by context, the terms “a”, “an”, and “the” mean “one or more.” For example, “a molecule” should be interpreted to mean “one or more molecules.” As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method comprising predicting one or more properties of a chemical compound:
generating a 3D molecular input point set from compound information with a computer system, wherein each atom point of the 3D molecular input point set comprises x, y, z-coordinates and one or more atomic attributes;
convoluting the 3D molecular input point set to generate a layer with the computer system, wherein convoluting an input feature matrix generates a dₒᵤₜ×n feature matrix, where the input feature matrix is a dᵢₙ×n feature matrix, n is the number of atoms in the compound, and dᵢₙ comprises the x, y, z-coordinates and the one or more attributes;
generating one or more additional layers by repeating the convolution step using the dₒᵤₜ×n feature matrix as the input matrix with the computer system;
encoding the chemical compound by stacking the generated layers with the computer system; and
generating a report comprising one or more predicted properties of the encoded chemical compound.

2. The method of claim 1, wherein the encoded chemical compound is permutation invariant.

3. The method of claim 1, wherein each generated layer comprises three subnetworks for atom feature extraction, neighbor feature extraction, and feature integration.

4. The method of claim 3, wherein
for each atom i with an input feature vector xᵢ (xᵢ∈ [symbol] ), a local subgraph is built for each atom that contains its k-nearest neighbors, whose feature vectors are denoted by yᵢ^{j} (j=1, 2, . . . , k);
through the neighbor feature extraction subnetwork, the k neighbor features (bᵢ^{j}, j=1, 2, . . . , k) are derived from the atom features xᵢ and the neighbor features yᵢ^{j}, and then concatenated to obtain a neighbor feature vector cᵢ by using a pooling operation (Σ);
through the atom feature extraction subnetwork, the atom feature vector aᵢ is derived from the atom features xᵢ; and
through the feature integration subnetwork, the atom and neighbor features are integrated into a latent feature vector xᵢ′ (xᵢ′∈ [symbol] ).

5. The method of claim 1, wherein the method further comprises multiplying an affine transformation matrix onto the x, y, z-coordinates prior to convolution.

6. The method of claim 5, wherein the multiplying the affine transformation matrix generates a rigid transformation invariant matrix.

7. The method of claim 1, wherein the encoded chemical compound is combined with meta data.

8. The method of claim 7, wherein the meta data comprises a precursor type or a collision energy.

9. The method of claim 1, wherein the report is generated by embedding the encoded chemical compound into a vector by fully connected and/or max-pooling layers.

10. The method of claim 1, wherein the one or more attributes comprises one or more of encoding of an atom type, number of immediate neighbors, valence, atomic mass, atomic charge, number of immediate hydrogen, aromaticity, and ring system.

11. The method of claim 1, wherein the report comprises a predicted mass spectra mass-to-charge-ratio (m/z) or a relative intensity at the predicted m/z.

12. The method of claim 1, wherein pretrained prediction model weights are used to initialize weights for a second, different prediction model.

13. The method of claim 12, wherein pretrained prediction model weights are mass spectrometry prediction model weights.

14. The method of claim 13, wherein the report comprises a predicted chemical property that is neither a mass spectra mass-to-charge-ratio (m/z) nor a relative intensity at the predicted m/z.

15. The method of claim 14, wherein the report comprises a predicted retention time, collisional cross section, solubility, or toxicity.

16. A computing device comprising:
a communication system or input that receives compound information,
a processor in communication with the communication system, the input, and memory, wherein the memory comprises machine-executable code that, upon execution by the processor, implements the method according to claim 1.

17. The system of claim 16, wherein the communications system receives pretrained prediction model weights.

18. A computer readable medium comprising machine-executable code that, upon execution by a processor, implements the method according to claim 1.
